(19) United States
(12) Patent Application Publication
(10) Pub. No.: US 2013/0011415 A1
(43) Pub. Date: Jan. 10, 2013

(54) MILK DERIVED ANTIGEN SPECIFIC ANTIBODIES FOR INDUCING AN ADAPTIVE IMMUNE RESPONSE, METHODS OF PREPARATION AND USES THEREOF

(76) Inventor: Ruprecht Jules Joost van Neerven, Wageningen (NL)

(21) Appl. No.: 13/521,919

(22) PCT Filed: Jan. 14, 2011
(86) PCT No.: PCT/NL2011/050025

§ 371 Date: Aug. 30, 2012

(30) Foreign Application Priority Data
Jan. 15, 2010 (NL) 2004099

(51) Int. Cl. A61K 39/395; A61P 37/04; A61P 29/00; A61P 37/06; A61P 31/00; C07K 16/00; A61P 37/08

(52) U.S. Cl. 424/171.1; 424/130.1; 530/387.1

(57) ABSTRACT

The invention relates to the field of antibodies. In particular the invention relates to milk-derived antigen specific antibodies for generating an adaptive immune response, methods of preparation, and uses thereof.

### FIELD OF THE INVENTION

The invention relates to the field of antibodies. In particular the invention relates to milk-derived antigen specific antibodies for generating an adaptive immune response, methods of preparation, and uses thereof.

### BACKGROUND OF THE INVENTION

The immune system in higher organisms, such as mammals, is specialized to protect the organism from the consequences of exposure to foreign materials or external biological influences. The immune system may thus, for example, protect the body against bacterial or viral infection, or the effect of cancer cells or other foreign pathogenic substances. The immune system provides both an innate immunity and an immunity that can, adapt to infections or other challenges to the system. Innate immunity allows the organism to quickly respond to exposure to foreign organisms or materials. The adaptive immune system enables the organism to develop a long-term ability to respond to exposure to specific organisms or foreign materials. These two systems act to protect the body from challenges resulting from exposure to pathogenic organisms and foreign substances.

The present disclosure provides methods and compositions for enhancing and/or inducing the immune response, in particular, the adaptive immune response, in a individual.

### SUMMARY OF THE INVENTION

One aspect of the disclosure provides for ruminant, preferably bovine antigen-specific antibodies and their use for enhancing and/or inducing an adaptive immune response against an antigen in an individual to which the antibody has been administered.

Another aspect of the disclosure provides ruminant, preferably bovine antigen-specific antibody used for promoting the efficient presentation of the antigen to T-cells.

Another aspect of the disclosure provides a ruminant, preferably bovine antigen-specific antibody used for promoting the production of non-inflammatory, antigen-specific IgA in the individual. Preferably, the antigen-specific antibody is used for promoting a non-inflammatory immune response in the respiratory tract of the individual. Preferably, the antigen-specific antibody is used for promoting a non-inflammatory immune response in the intestine of the individual.

Another aspect of the disclosure provides a ruminant, preferably bovine antigen-specific antibody used for activating dendritic cells. Preferably, the dendritic cells produce one or more cytokines, preferably IL-10 and/or TNF-α.

Another aspect of the disclosure provides compositions suitable for oral administration in humans comprising said antibodies. Preferably the composition is a child nutritional composition, preferably infant formula.

Another aspect of the disclosure provides methods for enhancing and/or inducing an adaptive immune response in an individual, comprising conjointly administering to said individual in need thereof a ruminant, preferably bovine, antigen-specific antibody and said antigen.

Another aspect of the disclosure provides methods for promoting the efficient presentation of the antigen to T-cells in an individual, comprising conjointly administering to said individual in need thereof a ruminant, preferably bovine, antigen-specific antibody and said antigen.

Another aspect of the disclosure provides methods for promoting the production of non-inflammatory, antigen-specific IgA in an individual, comprising conjointly administering to said individual in need thereof a ruminant, preferably bovine, antigen-specific antibody and said antigen.

Another aspect of the disclosure provides methods for activating dendritic cells in an individual, comprising conjointly administering to said individual in need thereof a ruminant, preferably bovine, antigen-specific antibody and said antigen.

Preferably, the antigen-specific antibodies are obtained from the milk, milk product, colostrum, or colostrum product of a ruminant, more preferably from a bovine. Preferably, the antigen is present in the individual, more preferably in the gastro-intestinal tract or airways of the individual.

Preferably, the individual is human. Preferably, the antibody is obtained from bovine milk, milk product, colostrum, or colostrum product. Preferably, the antibody is formulated into a pharmaceutical or nutritional composition suitable for human consumption. Preferably, the composition is infant formula or growing-up milk. Preferably, the composition is a functional food. Preferably, the individual is afflicted with, or at risk to develop, an allergic disease, an infectious disease, an autoimmune disease, or an inflammatory disease. Preferably, the antigen is present in the milk, milk product, colostrum, or colostrum product.

Preferably, the collected milk or colostrum is tested for the presence of the antigen and/or the antigen-specific antibody. Preferably, the collected milk or colostrum is subjected to at least one processing step to enrich for the antigen-specific antibody. Preferably, the ruminant has not been immunized with said antigen prior to collecting the milk or colostrum.

In one aspect of the disclosure, methods are provided for delaying the onset or reducing the severity of one or more symptoms of a disorder selected from an allergic disease, an autoimmune disease, an infectious disease, and/or an inflammatory disease in an individual at risk of developing said disease, comprising providing said individual at risk thereof with a ruminant, preferably bovine, antigen-specific antibody. Preferably, the individual is conjointly administered said antigen. Preferably, the antigen-specific antibody is obtained from bovine milk, milk product, colostrum, or colostrum product of a ruminant, more preferably a bovine.

The disclosure further provides the following embodiments.

1. Use of an antigen-specific antibody obtained from the milk, milk product, colostrum, or colostrum product of a ruminant, preferably a bovine, for enhancing and/or inducing an adaptive immune response against an antigen in an individual to which the antibody has been administered.

2. Use of an antigen-specific antibody obtained from the milk, milk product, colostrum, or colostrum product of a ruminant, preferably a bovine, for promoting the efficient presentation of the antigen to T-cells.

3. Use of an antigen-specific antibody obtained from the milk, milk product, colostrum, or colostrum product of a ruminant, preferably a bovine, for promoting the production of non-inflammatory, antigen-specific IgA in the individual.

4. Use of an antigen-specific antibody obtained from the milk, milk product, colostrum, or colostrum product of a ruminant, preferably a bovine, for activating dendritic cells to produce one or more cytokines, preferably IL-10 and/or TNF-α.

5. The use of any of embodiments 1-4 and 27, wherein the antigen is present in the individual.

6. The use of embodiment 5, wherein the antigen is present in the gastro-intestinal tract or airways of the individual.

7. The use of any of embodiments 1-6 and 27, wherein the individual is human.

8. The use of any of embodiments 1-7 and 27, for promoting a non-inflammatory immune response in the respiratory tract of the individual.

9. The use of any of embodiments 1-7 and 27, for promoting a non-inflammatory immune response in the intestine of the individual.

10. The use of any of embodiments 1-9 and 27, wherein the antibody is formulated into a pharmaceutical or nutritional composition suitable for human consumption.

11. The use of embodiment 10 and 27, wherein the composition is infant formula or growing-up milk.

12. The use of embodiment 10, wherein the composition is a functional food.

13. The use of any of embodiments 1-12 and 27, wherein the individual is afflicted with or at risk of developing an allergic disease, an autoimmune disease, an infectious disease, or an inflammatory disease.

14. The use of any of embodiments 1-13 and 27, wherein the antigen is present in the milk, milk product, colostrum, or colostrum product.

15. The use of any of embodiments 1-14 and 27, wherein the collected milk or colostrum is tested for the presence of the antigen and/or the antigen-specific antibody.

16. The use of any of embodiments 1-15 and 27, wherein the collected milk or colostrum is subjected to at least one processing step to enrich for the antigen-specific antibody.

17. The use of any of embodiments 1-16 and 27, wherein the ruminant has not been immunized with said antigen prior to collecting the milk or colostrum.

18. A method for enhancing and/or inducing an adaptive immune response in an individual, comprising conjointly administering to said individual in need thereof a ruminant, preferably bovine, antigen-specific antibody and said antigen.

19. The method of embodiment 18, wherein said individual is afflicted with or at risk of developing an allergic disease, an autoimmune disease, an infectious disease, and/or an inflammatory disease.

20. A method for delaying the onset or reducing the severity of one or more symptoms of a disorder selected from an allergic disease, an autoimmune disease, an infectious disease, and/or an inflammatory disease in an individual at risk of developing said disease, comprising providing said individual at risk thereof with a ruminant, preferably bovine, antigen-specific antibody.

21. The method of embodiment 20, wherein the individual is conjointly administered said antigen.

22. The methods of any of embodiments 18-21, wherein the antigen-specific antibody is obtained from milk, milk product, colostrum, or colostrum product of a ruminant, preferably a bovine.

23. Antigen-specific antibody obtained from the milk, milk product, colostrum, or colostrum product of a ruminant, preferably a bovine, for use in enhancing and/or inducing an adaptive immune response against the antigen in an individual to which the antibody has been administered.

24. Antigen-specific antibody obtained from the milk, milk product, colostrum, or colostrum product of a ruminant, preferably a bovine, for use in activating dendritic cells to produce one or more cytokines, preferably IL-10 and/or TNF-α.

25. Child nutritional composition, preferably infant formula, comprising antigen-specific antibody obtained from the milk, milk product, colostrum, or a colostrum product of a ruminant, preferably a bovine, for use in enhancing and/or inducing an adaptive immune response against the antigen in a child, preferably an infant, to which the antibody has been administered.

26. Child nutritional composition, preferably infant formula, comprising antigen-specific antibody obtained from the milk, milk product, colostrum, or a colostrum product of a ruminant, preferably a bovine, for use in activating dendritic cells to produce one or more cytokines, preferably IL-10 and/or TNF-α.

27. Use of an antigen-specific antibody obtained from the milk, milk product, colostrum, or colostrum product of a ruminant, preferably a bovine, for the preparation of a medicament for one of more of the following a) enhancing and/or inducing an adaptive immune response against an antigen in an individual to which the antibody has been administered b) for promoting the efficient presentation of the antigen to T-cells, c) for promoting the production of non-inflammatory, antigen-specific IgA in the individual, and d) for activating dendritic cells to produce one or more cytokines, preferably IL-10 and/or TNF-α.

28. The child nutritional compositions of embodiments 25-25, wherein the composition comprises carbohydrates preferably present between 25-50% wt, more preferably 28-46% wt, fat free milk solids preferably present between 12-20% wt, more preferably between 15-18% wt, fat preferably present between 22-30% wt, more preferably between 24-27% wt, and WPC-35 preferably present between 5-20% wt, more preferably between 8-18%.

29. The child nutritional compositions of embodiment 28 further comprising fucosyllactose, preferably between 0.01-0.5% wt, more preferably between 0.15-0.3; siallyllactose, e.g., vivinal siallyllactose, preferably between 1-6wt %, more preferably between 2-5wt %; and/or galacto-oligosaccharide (GOS), e.g. vivinal galacto-oligosaccharide, preferably between 8-15wt %, more preferably between 10-13wt %.

30. The child nutritional compositions of embodiment 28 or 29 comprising colostrum, preferably between 1-5% wt colostrum, more preferably between 2-4% wt colostrum.

### DETAILED DESCRIPTION OF THE INVENTION

Mother's milk and cow's milk contains antibodies that are specific for a wide range of potential pathogenic microorganisms, viruses and other antigens. These antibodies protect the infant that is not yet able to mount an effective immune response against infectious agents that the mother has already been exposed to. IgA is the predominant antibody in human milk, but IgG₁ is the predominant antibody isotype found in bovine colostrum and milk. The IgA in human milk is generally known to exert its effect in the intestine, whilst the mother transfers IgG to the baby via the placenta before birth.

As ruminants produce milk that contains antibodies, the milk or antibody preparations thereof are considered for use in humans to supplement or replace the function of mother's milk. The ruminant antibodies are intended to bind to and neutralize or otherwise impair the human pathogen in the intestine of individuals ingesting the antibodies and convey passive immunity.

In contrast to short-lived passive immunity, adaptive immune responses occur during the lifetime of an individual as an adaptation to infection with that pathogen, and in many cases, confer lifelong protective immunity to reinfection with the same pathogen. An adaptive immune response involves activation, selection, and clonal proliferation of two major classes of lymphocytes termed T cells and B cells. After encountering an antigen, T cells proliferate and differentiate into antigen-specific effector cells, while B cells proliferate and differentiate into antibody-secreting cells.

Adaptive immune responses are initiated by activation of CD4+ T helper cells after antigen presentation by antigen presenting cells (APC). APC take up exogenous (protein) antigens from their environment via pinocytosis or receptor-mediated endocytosis, degrade them into peptides, and present these peptides in the context of MHC class II molecules to antigen-specific T cells.

Facilitated antigen presentation has been described for cell surface expressed Fc receptors on professional antigen presenting cells. Fc receptors are transmembrane proteins that specifically bind to the constant region of circulating immunoglobulins. Fc receptors can internalize bacteria and viruses as well as protein antigens that are bound by circulating serum immunoglobulins, resulting in killing of pathogenic microorganisms and antigen presentation to initiate immune responses.

Low- and intermediate affinity Fc receptors have a low affinity for monomeric immunoglobulins, but have a high affinity for immune complexes. As a result, these receptors will only bind immunoglobulins when they are in complexes together with the antigens they recognize. Indeed, for Fcγ receptors (Celis et al., Proc Natl Acad Sci U S A. 1984; 81:6846-50, Gosselin et al., J Immunol. 1992;149:3477-81), Fcα receptors (Shen et al Blood. 2001;97:205-13), and Fcε receptors (Santamaria et al., Hum Immunol. 1993;37:23-30) it has been shown that immune complexes are taken up more efficiently by antigen presenting cells than the antigens alone. These studies demonstrate that antigen-specific responses of CD4+ T helper cells are facilitated strongly by specific antigen uptake via Fc receptors on antigen-presenting cells, resulting in more efficient immune responses, especially when low antigen doses are present.

Two other, nonclassical, Fc receptors are the polymeric IgA receptor (pIgR) and the neonatal Fc receptor (FcRN). Both receptors specialize in the transport of immunoglobulins over epithelial sites. PIgR is expressed on M cells and on epithelia, and FcRN is expressed on epithelia, but also on antigen presenting cells. Recent evidence suggests that these receptors play a crucial role in sampling the intestinal lumen for immune complexes, and to transport these immune complexes into the underlying lymphoid tissues, where efficient immune responses are mounted to the pathogens bound to immunoglobulins present in the lumen.

Another Fc receptor that can transport luminal antigens into intestinal lymphoid structures is CD23, the low affinity IgE receptor. CD23 is expressed on intestinal epithelium, and has been shown to play a role in the internalization of food allergens in animal models (Yang et al,. J Clin Invest. 2000;106:879-86).

The present invention is based, in part, on the discovery that bovine immunoglobulins are able to bind to and to activate human immature dendritic cells (DC) in the presence of antigen (see Examples 1 and 2). Intestine mucosal DC have been reported to open tight junctions between epithelial cells and send their dendrites outside the epithelium to sample the gut lumen. Furthermore, immune complexed antigens that are internalized via classical as well as non-classical Fc receptors on antigen presenting cells are known to be presented far more efficiently to T lymphocytes than non-complexed antigens (so-called ‘vaccine effect’).

While not wishing to be bound by theory, we propose that human mucosal DC can be activated by immune complexes comprising ruminant antigen-specific antibodies and antigens, e.g., from ingested harmful microbial pathogens.

Thus, one aspect of the invention provides methods for enhancing and/or inducing adaptive immunity in an individual using antigen-specific antibodies derived from milk of a ruminant, preferably a bovine. Preferably, the antigen-specific antibody promotes the production of human immunoglobulins upon challenge with an antigen, such as a pathogenic microorganism, virus, or a food- or inhalation allergen. Preferably, the antigen-specific antibody promotes the efficient presentation of an antigen to T-cells. While the individual can be any mammalian species, human is preferred. Preferably, the antigen-specific antibody is non-denatured.

Bovine immunoglobulins isolated from milk can specifically recognize LPS from gram negative pathogenic bacteria like E. coli and Salmonella, as well as common allergens such as house dust mite, birch pollen, and grass pollen (see PCT Publication WO2008/127105, which is hereby incorporated by reference in its entirety). In addition, bovine immunoglobulins isolated from milk can also recognize peptidoglycan from gram positive microorgansims, gastrointestinal viruses, and bacterial toxins.

Upper airway pathogens are swallowed in considerable quantities, and thus are available in the intestine. Without being bound by theory, it is believed that antigens, whether swallowed or present in the airways, are bound by bovine immunoglobulins, resulting in targeted uptake and presentation of the antigen-immunoglobulin complex through classical and non-classical Fc receptors. Antigen presentation results in immune responses to these antigens, such as, e.g., the production of IgA immunoglobulins in the intestinal and airway mucosa. As a result, individuals that receive bovine immunoglobulins, for example by oral administration, may be more efficient in producing protective IgA to these intestinal and airway pathogens. Prophylactic administration of bovine IgG thus induces adaptive immunity against a particular antigen.

Preferably, the antigen-specific antibody promotes the production of a non-inflammatory immune response, e.g., in the respiratory and/or gastro-intestinal tract (preferably intestine) of the individual to which the antibody is administered. The ruminant antibody may promote the production of any type of human immunoglobulin. Preferably, the production of IgA is promoted.

In some embodiments, the antibodies described herein activate human dendritic cells (DC), and, preferably, induce cytokine production, preferably IL-10 and/or TNF-α, in said human DC. In some embodiments, the antibodies described herein activate human DCs and, preferably induce cytokine production, preferably IL-10 and/or TNF-α, production in said human DC in the presence of an antigen, such as, e.g., LPS.

In a preferred embodiment, said ruminant antibody is of type IgG₁, IgA or a combination thereof. Preferably, said antibody is of the IgG₁ type or composition comprising both IgG₁ and IgA specific for said antigen.

Preferably, the antigen recognized by the ruminant antibody is present in the individual to which the antibody is administered. Thus, the individual may have been exposed to the antigen, for example by swallowing and/or inhaling a pathogen, pathogen debris, or allergen.

In some embodiments, the antigen may be administered to the individual. For example, the collected milk comprising the antibody may contain the antigen or the antigen may be added to a preparation, such as a nutritional or pharmaceutical composition, comprising the antibody. Preferably, the antigen is present in the gastrointestinal tract or airways of the individual.

One aspect of the disclosure, therefore, provides methods for enhancing and/or inducing an adaptive immune response in an individual, comprising conjointly administering to said individual in need thereof a ruminant antigen-specific antibody and said antigen. In some embodiments, said individual is afflicted with or at risk of developing an allergic disease, an infectious disease, an autoimmune disease, and/or an inflammatory disease.

As used herein, the phrase “conjoint administration” refers to any form of administration of two or more different compounds such that the second compound is administered while the previously administered compound is still effective in the body (e.g., the two compounds are simultaneously effective in the patient, which may include synergistic effects of the two compounds). For example, the different compounds can be administered either in the same formulation or in a separate formulation, either concomitantly or sequentially.

Another aspect of the disclosure provides methods for delaying the onset or reducing the severity of one or more symptoms of a disorder selected from an allergic disease, an infectious disease, an autoimmune disease, and/or an inflammatory disease in an individual, comprising providing said individual with a ruminant antigen-specific antibody. In some embodiments, the methods prevent and/or reduce the risk of the individual from developing a disorder selected from an allergic disease, an infectious disease, an autoimmune disease, and/or an inflammatory disease. In some embodiments, the individual is at risk of developing said disease. Individuals at risk of developing said disease include the elderly, adults, adolescents, children, and infants. In some embodiments, the individual is conjointly administered said antigen. While not wishing to be bound by theory, administration of a ruminant antigen-specific antibody induces an adaptive immune response against said antigen. The production of antibodies, in particular IgA antibodies, by the individual against said antigen is thought to neutralize the effect of said antigen upon subsequent exposure.

In one aspect of the disclosure, a ruminant antibody useful for the methods described herein is specific for an allergy antigen. While not wishing to be bound by theory, the production of a non-inflammatory immune response, preferably via antigen-specific IgA, is thought to neutralize the effect of allergens. In some embodiments, the antigen specific antibody is specific for a food allergy-associated antigen, preferably a food allergy associated antigen from milk, such as cow's milk, soy, hazelnut, egg or apple. In a particularly preferred embodiment said allergy antigen is an allergy-associated peanut antigen. In another embodiment said allergen is an inhalation allergen or a venom allergen selected from but not limited to the group of grass pollen allergens, tree pollen allergens, house dust mite allergens, cat allergens, mold or fungus allergens, or stinging insect venoms. Said inhalation and venom allergens can enter the human body via the respiratory tract or via a sting of an insect such as a bee or a wasp.

In a preferred embodiment, an individual administered a ruminant antibody has developed or is at risk of developing an allergic diseases selected from the group of food allergy, rhinitis, conjunctivitis, asthma or venom allergy.

In one aspect of the disclosure, the methods provided for enhancing and/or inducing an adaptive immune response in an individual are useful for treating, preventing, or reducing onset or severity of symptoms of an infectious disease. In some embodiments, a ruminant antibody useful for the methods described herein is specific for a micro-organism or a virus, preferably with a tropism for the intestine or for the respiratory tract.

Preferably said micro-organism is selected from a member of the genus Escherichia, preferably Escherichia coli, a member of the genus Salmonella, preferably Salmonella typhimurium, a member of the genus Campylobacter, preferably Campylobacter jejuni, a member of the genus Helicobacter, preferably Helicobacter pilori, a member of the genus Bordotella, preferably Bordotella pertussis, a member of the genus Clostridium, a member of the genus Shigella, a member of the genus Streptococcus, preferably Streptococcus pneumoniae, a member of the genus Staphylococcus, preferably Staphylococcus aureus, a member of the genus Haemophilus, preferably Haemophilus influenzae, a member of the genus Pseudomonas, preferably Pseudomonas aeruginosa, a member of the genus Moraxella, preferably Moraxella catarrhalis, a member of the genus Chlamydia, preferably Chlamydia pneumoniae, a member of the genus Mycoplasma, preferably Mycoplasma pneumoniae or a member of the genus Legionella, preferably Legionella pneumophila. Preferred virus specific antibodies are directed against a member of the genus influenza, preferably influenza virus A or influenza virus B, a member of the parainfluenza viruses, a member of the respiratory synctial virus, a member of the rotaviruses, a member of the noroviruses, a member of the enteroviruses, a member of the cytomegaloviruses or a member of the adenoviruses.

In some embodiments, the ruminant antibody is specific for a proinflammatory cytokine or a stimulatory signalling factor produced by immune cells of the individual and that contribute to the maintenance and/or severity of a symptom of an autoimmune disease, inflammatory disease, or otherwise over-active immune system. Thus preferably an antibody of the invention is specific for a factor excreted by immune cells. In a particularly preferred embodiment, a ruminant antibody useful in the methods of the invention is specific for human TNF-α, IL-12p70, IL-17, IFN-gamma or human IL-23. In yet another embodiment said antibody is specific for an immune cell, an epithelial cell, an intraepithelial cell, an antigen presenting cell or an M-cell of the individual to be treated, preferably a T-cell, a B-cell or a dendritic cell. In a preferred embodiment, said individual is suffering from inflammatory bowel disease. In a further preferred embodiment said bovine antibody is specific for an antigen of a microorganism associated with said auto-immune disease.

The ruminant antibodies described herein can be administered to a human of any age. In some embodiments, the individual is between 0 and 24 months old, preferably between 0 and 12 months, more preferably between 6 and 24 months old (typically when solid food is included in the diet). Antibodies have a longer half life in the gastro-intestinal tract of infants of between 0-12 months old as compared to older children and adults. In some embodiments, the individual is at least 16, at least 20, at least 30, at least 40, at least 50, or at least 60 years old.

A ruminant antibody useful for the methods described herein may be provided in a preparation of a medicament for systemic treatment and/or for the prevention of a disease in an individual. In some embodiments, the medicament is for prophylactic treatment and reduces the severity and/or delays onset of one or more symptoms of said disease. Preferably said individual is a human. Preferably said medicament is for oral administration.

A ruminant antibody useful for the methods described herein may be provided in suitable compositions and formulations. Preferably said antigen specific antibody comprises less than 1% of the total amount of antibody in said composition. More preferably said antigen specific antibody comprises less than 0.1% of the total amount of antibody in said composition. In a preferred embodiment at least 15% and more preferably at least 25% and more preferably at least 35% and more preferably at least 50% and more preferably at least 60% and more preferably at least 75% even more preferably at least 80% of the antibodies are IgGT, IgA or a combination thereof. Preferably said composition is enriched for IgG₁ over IgA. In another preferred embodiment of the invention the composition is enriched for IgA over IgG₁.

In some embodiments, a composition comprises a ruminant antibody in an amount that is between 0.001 to 100% of the total amount of protein in said composition. Preferably said antibody is present in an amount of 0.1 to 50% of the total amount of protein in said composition. Preferably said antibodies are present in an amount of between about 5-30%, more preferably between 10-30% of the total amount of protein

In a preferred embodiment at least 10%, more preferably at 15% and more preferably at least 25% and more preferably at least 35% and more preferably at least 50% and more preferably at least 60% and more preferably at least 75% even more preferably at least 80% of the antibodies are IgG₁, IgA or a combination thereof. Preferably said composition is enriched for IgG₁ over IgA. In another preferred embodiment of the invention the composition is enriched for IgA over IgG₁.

In some embodiments, the ruminant antibodies described herein are provided as a preparation suitable for administration via intramuscular injection, intravenous administration, or via the rectum or vagina.

Preferably, the ruminant antibodies described herein are provided as a preparation for oral administration. The ruminant antibodies can be used in any nutritional or pharmaceutical composition, such as dietary, food, or health supplements; concentrate; and functional food (milk or other dairy product, protein bars, sports nutrition, drinks, food supplement, food for medical purposes, clinical nutrition, and preferably products for child nutrition). Functional food refers to any fresh or processed food comprising one or more components that provide a physiological benefit to the consumer of the food. In some embodiments, the one or more components are not normally present in the functional food; in some embodiments, the one or more components are normally present, but are present at a higher level in the functional food. By way of example, as defined herein, raw milk is not a functional food, whereas milk that has been processed to concentrate immunoglobulins, or milk that has been supplemented with immunoglobulins is a functional food. The physiological benefit may be a result of the one or more components alone or the combination of the one or more components and other constituents of the food. Preferably, the functional food comprising the bovine antibodies according to the invention is dairy produce or dairy product, in particular a cheese, butter, yogurt, kefir, quark, cultured milk, buttermilk, cream, evaporated milk, dried milk, whey, lactose, milk protein, flavored milk, low-fat milk, flavored whey or dairy fat product or preparation. In some embodiments, the functional food is milk, a processed colostrum product, or a processed milk product. In some embodiments, the functional food comprises at least 100 μg/ml, at least 200 μg/ml, at least 300 μg/ml at least 500 μg/ml at least 700 μg/ml, at least 1 mg/ml, or at least 2 mg/ml of antigen specific bovine immunoglobulins.

A processed milk product refers to a food product obtained from milk, that has been subjected to at least one processing step (such as, e.g., any pasteurization method known to one skilled in the art) that reduces the number of viable pathogens in the product as compared to the non-processed food product.

Child nutrition includes infant formulas, follow-on formulas, infant cereal products or growing-up milk, i.e. modified milk or milk powder suitable for children of 1-3 years. The term “infant” as used herein, refers generally to children less than about 2 years of age, most typically less than about 1 year of age, and the term “infant formula” refers to compositions administered to infants to meet their sole, primary, or supplemental nutritional needs. Infant formula comprises lipids (such as tri, di, and monoglycerides, phospholipids, sphingolipids, and fatty acids), proteins, and carbohydrates (such as disaccharides, monosaccharides, maltodextrins, and starch), and preferably further comprises vitamins and minerals. Infant formulas and methods of making are well known to one skilled in the art and are described in, e.g., U.S. Pat. No. 7,090,862.

Preferably infant formula also comprises one or more of whey protein concentrate 35 (WPC 35; Whey protein concentrate 35% Protein on dry matter), galacto-oligosaccharide (GOS), fructooligosaccharides (FOS), (preferably in a ration of 9:1 GOS:FOS) Siallyllactose (SL), Fucosyllactose (FL), long chain polyunsaturated fatty acid, linoliec acid, alpha-linolenic acid, oleic acid, Arachidonic acid, Docosahexaenoic acid, Eicosapentaenoic acid, nucleosides, nucleotides, and hydrolyzed protein. Infant formula is provided comprising ruminant antibodies present in an amount of between 0.1% and 50%, preferably between 5% and 30%, even more preferably between 10% to 20% of the total amount of protein. In some embodiments, the bovine antibodies are present in an amount of between 0.5% and 5% of the dry weight, preferably between 1% to 2% of the dry weight. In some embodiments, reconstituted dry infant formula or liquid infant formula comprises between 0.01 to 10 mg/ml, preferably between 0.05 to 10 mg/ml, more preferably between 0.5 to 5 mg/ml, preferably between 1 to 2 mg/ml, or most preferably between 0.5 and 1 mg/ml of bovine antibodies. In a preferred embodiment, infant formula comprises between 1 and 10, preferably between 2 and 5, more preferably between 3 and 4 grams of colostrum per 100 gram of infant formula powder.

The disclosure further provides child nutritional compositions, preferably infant formula, comprising antigen-specific antibody obtained from the milk, milk product, colostrum, or a colostrum product of a ruminant, preferably a bovine, based on a composition of carbohydrates, fat free milk solids, fat, and WPC-35. Preferably, the carbohydrates are present at between 25-50% wt, preferably 28-46% wt, the fat free milk solids are present at between 12-20% wt, preferably between 15-18% wt, fat is present between 22-30% wt, preferably between 24-27% wt, and WPC-35 is present between 5-20% wt, preferably between 8-18%. These concentrations can be adjusted as needed by one of skill in the art. The compositions may further contain, either alone or in any combination, colostrum, preferably between 1-5% wt colostrum, more preferably between 2-4% wt colostrum; fucosyllactose, preferably between 0.01-0.5% wt, more preferably between 0.15-0.3; siallyllactose, e.g., vivinal siallyllactose, preferably between 1-6wt %, more preferably between 2-5wt %; or galacto-oligosaccharide (GOS), e.g. vivinal galacto-oligosaccharide, preferably between 8-15wt %, more preferably between 10-13wt %.

Preferably, the composition comprises colostrum and has the following composition: between 42-48% wt carbohydrates, preferably between 43-47% wt carbohydrates; between 14-18% wt fat free milk solids, preferably between 15-17% wt fat free milk solids; between 23-28% wt fat, preferably between 24-27% wt fat; between 5-15% wt WPC-35, preferably between 8-12% wt WPC-35, and between 1-5% wt colostrum, preferably between 2-4% wt colostrum.

Preferably, the composition comprises colostrum and GOS and has the following composition: between 30-35% wt carbohydrates, preferably between 32-35% wt carbohydrates; between 14-18% wt fat free milk solids, preferably between 15-17% wt fat free milk solids; between 23-28% wt fat, preferably between 24-27% wt fat; between 5-15% wt WPC-35, preferably between 8-12% wt WPC-35, between 1-5% wt colostrum, preferably between 2-4% wt colosturm, and between 8-15wt %, preferably between 10-13wt % GOS.

Preferably, the composition comprises colostrum , fucosyllactose, siallyllactose, and GOS has the following composition: between 25-35% wt carbohydrates, preferably between 28-32% wt carbohydrates; between 14-18% wt fat free milk solids, preferably between 14-17% wt fat free milk solids; between 23-28% wt fat, preferably between 24-27% wt fat; between 5-15% wt WPC-35, preferably between 8-12% wt WPC-35, between 1-5% wt colostrum, preferably between 2-4% wt colostrum, between 0.01-0.5% wt fucosyllactose, preferably between 0.15-0.3, siallyllactose between 1-6wt %, preferably between 2-5wt %; and galacto-oligosaccharide (GOS) between 8-15wt %, preferably between 10-13wt %. Exemplary formulas are depicted in FIG. 5.

The % wt definitions as disclosed herein are based on the total dry weight. The composition may therefore be a solid, e.g., powder, or may also be a liquid, such as ready-to-use infant formula.

Any source of IgG may be used to prepare child nutritional compositions as disclosed herein. The colostrum in the compositions disclosed above may be replaced partially or entirely by other sources of IgG, such as milk or whey, or by purified IgG. As shown in Table 2 of Example 5, raw milk contains more than 200 μg/ml of IgG1. Four liters of raw milk thus contains sufficient IgG to prepare a composition having an IgG content of around 0.78 g/l. Preferably the compositions when prepared as a liquid or reconstituted as a liquid comprise between 35-60g/l carbohydrates, more preferably between 37-58 g/l, even more preferably between 48-58 g/l; between 10-15 g/l protein, more preferably between 11-13 g/l; between 23-29 g/l fat, preferably between 25-27 g/l; between 3-10wt % of minerals and other components, preferably between 4-9g/l, and between 0.1-5g/l. These compositions may also contain siallyllactose, preferably between 0.1-0.3 g/l; fucosyllactose, preferably between 0.1-0.3 g/l fucosyllactose; and GOS, preferably between 3-6 g/l.

An orally delivered ruminant antibody preparation can be formulated in a fluid such as a watery solution, milk, yoghurt, or as a spray- or freeze-dried protein preparation, or encapsulated to protect the antibody in the stomach. Preferably, such antibody is added to an infant formula or a milk product meant for human consumption. In another preferred embodiment of the invention, the compositions described herein comprise a probiotic bacteria, or a prebiotic. The probiotic may be, for example, lactic acid bacteria such as Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Bifidobacterium longum, Bifidobacterium infantis, Bifidobacterium bifidum, and Pediococcus cerevisiae, or mixtures thereof. Said prebiotic can be any prebiotic oligosaccharide, such as galactooligosaccharide, fructo-oligosaccharides, fuco-oligosaccharides, sialo-oligosaccharides, as well as inulin, beta glycans, carob four, gums, pectins, sialyllactone, galactans, and nucleotides.

Although ruminant antibodies are relatively resistant to degradation in the human gastro-intestinal tract, it is preferred that said preparation for oral administration comprises a delivery vehicle comprising said antigen specific antibody, wherein said delivery vehicle enables targeted intestinal release of said antibody from said vehicle. Various delivery vehicles are known in the art that allow specific release of enclosed or enveloped substances in the intestinal tract. It is preferred that said vehicles allow release in the small intestine. Bovine immunoglobulins may be formulated, for example, such that it is coated with an enteric coating which is resistant to dissolution in acidic conditions. Pharmaceutically acceptable excipients for enteric coating being insoluble at gastric pH but soluble at intestinal pH include cellulose acetate phthalate, hydroxpropylmethyl-cellulose phthalate, polyvinylacetate phthalate and acrylic acid polymers.

The release profile of a ruminant immunoglobulin composition can be altered by varying the coating thickness as well as the composition of the coating. If sustained release of bovine immunoglobulin is desired, sustained release coatings, or mixtures of sustained release coatings with enteric coatings, can be applied. Pharmaceutically acceptable excipients for sustained release coatings include cellulose derivatives and copolymers of methacrylic acid esters such as ethylcellulose and Eudragit NE, Eudragit RL and Eudragit RS. In some embodiments, sustained release formulations release 5% to 20% by weight of active substance within 1 h., 23% to 27% within 2 hr., 33% to 42% within 3 hrs, 75% to 82% within 5 hrs, 93% to 97% within 7 hrs, and 100% within 12 hrs. It is within the purview of one skilled in the art to modify the release profile based on the disorder to be treated.

Preferably, ruminant antibodies are present in a composition in such an amount that between 5 mg to 100 g per day is administered. In some embodiments, the bovine antibodies are present in child nutrition, such as infant formula, which is administered in an amount of between 20 mg to 10 g, preferably between 50 mg to 2 g, per day. Preferably, the child nutrition will be a liquid and will be administered in an amount of between 20 ml to 1000 ml per day. The dosage will depend on the age and weight of the individual.

In some embodiments, ruminant immunoglobulins are provided in combination with an anti-inflammatory cytokine, such as, e.g., TGF-β, preferably bovine TGF-β, for the use in modulating an immune response, preferably a pro-inflammatory or allergic immune response, as described herein. In some embodiments, the ruminant immunoglobulins and TGF-β are co-formulated into a single composition.

In some embodiments, the pharmaceutical or nutritional compositions described herein comprise a antigen-specific antibody and an antigen. Preferably, the antigen is specific for an allergen or pathogen as described herein.

Preferably, the ruminant antibodies described herein are bovine antigen-specific antibodies.

It was previously shown that milk from a ruminant that was not previously immunized for an antigen can specifically react with said antigen (PCT Publication WO2008/127105 and Collins et al., Int Arch Allergy Appl Immunol 1991; 96:362-367). This indicated that the milk contained antibodies that specifically bound the antigen in spite of the fact that the ruminant had not been immunized for said antigen. In the present disclosure, this aspect is exploited in an industrial setting where large volumes of milk are processed. In some embodiments, the disclosure thus provides ruminant antigen specific antibodies from milk, colostrum, or processed versions thereof, characterized in that the milk is derived from a mammal that has not been immunized with said antigen prior to collecting said milk.

The term antigen in the context of the present disclosure refers to an antigen of a human, such as antigens from pathogens able to infect and cause disease in a human and allergens to which a human can become allergic. “Immunization for an antigen” refers to administering a composition comprising the antigen and typically an adjuvant to a mammal with the intention of obtaining an immune response against said antigen, said adjuvant, or combination thereof in said mammal. The administration involves the facilitation of intensive contact between the components of the composition and the immune cells of the mammal. Such administrations typically involve depositing the composition inside the mammal by breaking the skin layer. Immunization thus means immunization of said ruminant with an antigen of a human, a pathogen able to infect and cause disease in a human, an allergen to which a human can become allergic or a combination thereof. Vaccination of said ruminant for an antigen of a pathogen of said ruminant to treat or prevent a disease in said ruminant is therefore not considered an immunization in the context of the present disclosure. Oral administration of antigen as, for instance (part of) the food of said ruminant, is often associated with the induction of tolerance in the mammal for said antigen and is therefore not considered an immunization in the present disclosure.

The ruminant antibodies as described herein may be collected directly from the milk or at later stages during milk processing from a milk product, such as from whey (e.g. milk whey, colostral whey, cheese whey and acid (casein) whey), skim milk or fermented milk, or reconstituted whey protein concentrate or powders. Ruminant antibodies may also be collected from colostrum at all stages, including the colostrum produced before birth, immediately after birth, or up to one month after birth; as well as a colostrum product, such as colostrum powder or colostrum whey protein concentrate.

In a preferred embodiment, a method for obtaining the antibodies described herein comprises collecting milk or colostrum from a lactating ruminant and collecting said antibody from said milk or colostrum. Preferably milk or colostrum of at least two individual ruminants of the same species is pooled prior to collecting said antibody. In this way, also low abundant antibodies can be collected in sufficient amounts. For this purpose it is preferred to collect antibodies from milk or colostrum of at least 10 and more preferably at least 100 individual ruminants preferably of the same species. Preferably said ruminant is a bovine, preferably a member of the genus Bos. Of the genus Bos, the commercially exploited species are preferred. Preferably said ruminant is of the species Gayal, Bos frontalis (domestic gaur), Bos mutus (Yak) or Bos taurus (Domestic Cattle). In another preferred embodiment, said ruminant is a goat or a sheep, preferably a member of the subfamily Caprinae. Of this subfamily the members of the genera Ovis or Capra are preferred. Of the genus Ovis, the species Ovis aries (Domestic Sheep) is preferred. Of the genus Capra the species Capra aegagrus hircus (the domesticated goat) is preferred. In another preferred embodiment, said ruminant is a camel, a donkey, a buffalo, a horse or a lama.

An antibody from an immunized or non-immunized ruminant is preferably specific for an allergen antigen, a human growth factor, a human antigen, a gastrointestinal or respiratory virus, a gastrointestinal or respiratory bacterium, a parasite, or other relevant diseases-related antigen. In one embodiment, said antibody is derived from an immunized ruminant. Preferably, said antibody is derived from a non-immunized ruminant.

As milk contains several components, notably fats, oils non-antibody protein and carbohydrates it is preferred to submit said milk to a processing step prior to collecting said antibody. A suitable starting preparation for the collection of antibodies is raw milk or colostrum. Milk or colostrum that is treated with heat to achieve a logarithmic kill of bacteria, a process referred to as pasteurization, can also be used. Pasteurisation typically uses temperatures below boiling since at temperatures above the boiling point for milk, casein micelles will irreversibly aggregate (or “curdle”). There are two main types of pasteurization used today: High Temperature/Short Time (HTST) and Extended Shelf Life (ESL) treatment. In the HTST process, milk is forced between metal plates or through pipes heated on the outside by hot water, and is heated to 71.7° C. (161 ° F.) for 15-20 seconds. ESL milk has a microbial filtration step and lower temperatures than HTST. Milk simply labeled “pasteurized” is usually treated with the HTST method. Milk can also be pasteurized at temperatures lower than 71.7° C. in combination with increased pressure. This is equally effective in logarithmic killing of bacteria as higher temperatures at lower pressure.

A milk processing step preferably comprises a separation step. A preferred separation step comprises a step wherein the milk or colostrum is (partially) depleted for fat. Thus another preferred starting preparation for the collection of antibodies comprises milk or colostrum that has undergone a fat depletion step. In a preferred embodiment said separation step comprises separating the milk in at least two parts and of which at least one is a protein rich part. Collection of antibody from batch processed milk or colostrum is possible, however, it is preferred that said processing step is part of a (semi)-continuous process. In a particularly preferred embodiment, a whey fraction is prepared from said milk and said antibody is collected from said whey. Thus in a preferred embodiment, the starting preparation for the collection of antibodies comprises whey. In yet another embodiment, the starting preparation for the collection of antibodies comprises fermented milk. A starting point can be as indicated herein above but also from a reconstituted concentrate or dry powder produced therefrom.

In a preferred embodiment, said ruminant is selected on a criterion for collection of said milk. This criterion includes time in the lactation cycle. In one embodiment, said ruminant is selected on the basis that it is producing normal milk and not colostrum. In another embodiment, said ruminant is selected on the basis that it is producing colostrum.

In a further preferred embodiment, said criterion comprises antibody content of the milk, antibody specificity in collected milk, type of food or type of food-supplement ingestion by said ruminant, vaccination for an antigen of a pathogen of said mammal (i.e., vaccination as previously described herein, such as vaccination against an animal disease), or a combination of two or more of said criteria.

In a preferred embodiment, said antibody is an IgG₁, IgA, IgG₂ or an IgM antibody. Preferably, said antibody is collected from an antibody-enriched fraction obtained from said milk. Preferably, said method further comprises a step to enhance the levels of IgG₁, IgA or a combination thereof when compared to other proteins present in the antibody enriched fraction. Preferably, the amount of beta-lactoglobulin is decreased by at least 50%. This can be accomplished for example by a specific hydrolysis of beta-lactoglobulin, or by removal of beta-lactoglobulin by size separation or by removal using an antibody specific for beta-lactoglobulin. Thus preferably, a method for collecting a bovine antibody further comprises a step to reduce the amount of beta-lactoglobulin in the antibody enriched fraction and/or an affinity purification step to obtain an antibody fraction that is enriched for said antigen specific antibody. Having the possibility to collect antigen specific antibody from a large collection of different ruminants of the same species allows the affinity purification of significant amounts of affinity purified antigen specific antibody, in spite of a possible low prevalence of said antibody in each individual milk sample.

In a preferred embodiment, the collected milk or colostrum is tested for either the presence of an antigen or the presence of antigen-specific antibodies. The overall content of immunoglobulins in the milk or colostrum can be determined and/or the presence of an antibody specific for a particular antibody. The milk or colostrum can be tested at any time during the processing steps, preferably after collection.

Regardless of the method used to obtain antigen-specific antibodies, the antibodies useful in the methods described herein are “functional”, i.e., are non-denatured antibodies that are capable of binding antigen. Therefore, preferred methods for obtaining antibodies do not significantly denature the antibodies.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

### DESCRIPTION OF THE FIGURES

FIG. 1A-C: Binding of bovine IgG (A), of bovine IgA (B), and of bovine IgM (C) from colostrum (grey histograms) and purified bovine immunoglobulins (black histograms) on human monocyte-derived immature DC. Dotted black histograms represent background staining. Data from one representative donor of two is shown.

FIG. 2A-B: Binding of human monomeric (grey histogram) and aggregated (mimicking immune complexes; black histogram) IgG on human monocyte-derived immature DC (A). The effect of pre-incubation with a blocking anti-CD32 antibody (10 μg/mL mouse anti-human CD32 clone FL18.26; BD Pharmingen) on the binding of human aggregated IgG (black histogram without anti-CD32; grey histogram with anti-CD32) on immature DC (B). Dotted (without anti-CD32) and solid (with anti-CD32) black histograms represent background staining.

FIG. 3A-B: Effect of colostrum, purified bovine immunoglobulins, and of human monomeric IgG—with or without 100 ng/mL LPS—on IL-10 (A) and TNF-α (B) production from human monocyte-derived immature DC after 2 days. Non-stimulated immature DC and 100 μg/mL poly I:C treated immature DC were run as negative and positive controls respectively (see bars (e) and (g)). Data from one representative donor of three is shown.

FIG. 4: Survival of bovine immunoglobulins after in vitro digestion experiments mimicking the conditions of the human GI tract. The percentage of IgG and IgA was determined after 0, 120, 180, and 240 minutes (t=time) of treatment mimicking infant digestion. The percentage of IgG and IgA was determined after 0, 60, 120, and 180 minutes of treatment mimicking adult digestion.

FIG. 5: Exemplary compositions for the preparation of infant formula.

All patent and literature references cited in the present specification are hereby incorporated by reference in their entirety.

The invention is further explained in the following examples. These examples do not limit the scope of the invention, but merely serve to clarify the invention.

### EXAMPLES

### Example 1

### Binding Capacity of Bovine Immunoglobulins on Immature Dendritic Cells

Bovine immunoglobulins were purified from bovine colostrum using an AFFI-T™ column followed by a proteinG column. This purified bovine immunoglobulin material was enriched for IgG1 but still contained IgM and IgA. The bovine colostrum and purified bovine immunoglobulins preparations contained IgG1>IgM>IgA>IgG2 (table 1).

TABLE 1
Bovine IgG1, IgG2, IgA, and IgM concentration
(mg/ml) in colostrum and AFFI-T ™/proteinG-
purified bovine immunoglobulins (mean ± sd; n = 2)

| | IgG₁ | IgG2 | IgA | IgM |
|---|---|---|---|---|
| Colostrum | 2.46 ± 1.11 | 0.00 | 0.31 ± 0.04 | 1.23 ± 0.37 |
| purified bovine | 5.93 ± 2.23 | 0.00 | 0.10 ± 0.02 | 0.94 ± 0.11 |
| immunoglobulins | | | | |

Human monocyte-derived immature DC expressing CD32/FcγR-II were incubated with bovine colostrum or purified bovine immunoglobulins under metabolic inactive conditions (4° C. and a non-toxic concentration of NaN3) to determine possible cross-species FcR/immunoglobulin interactions.

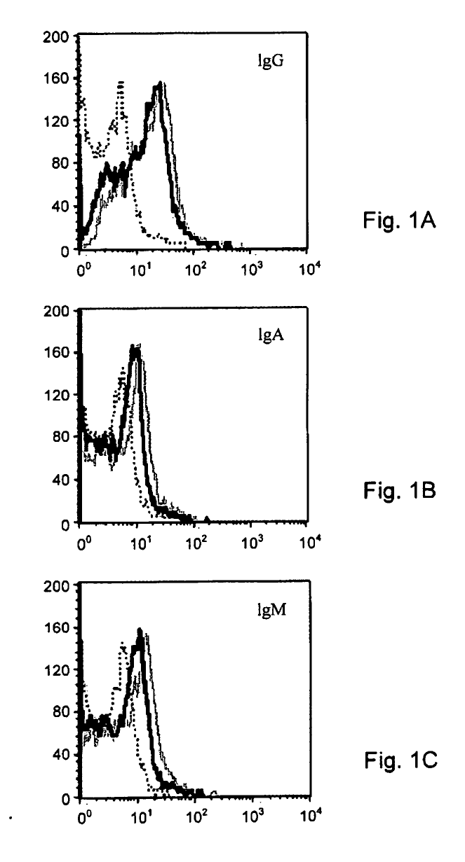

The binding of bovine IgG (250 μg/mL) from both colostrum and purified immunoglobulins preparations was clearly increased on immature DC (see FIG. 1). This bovine IgG binding was dose dependent. The binding of bovine IgA and IgM was only minimal.

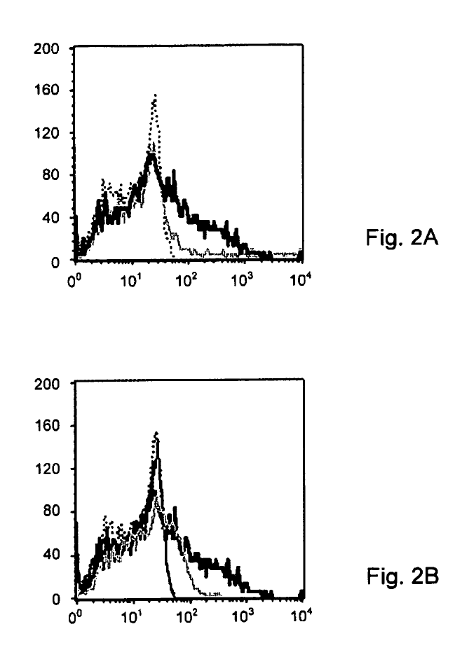

Monomeric human IgG (250 μg/mL) was not able to bind to CD32-expressing human immature DC, whereas aggregated human IgG (250 μg/mL) clearly bound to these cells (see FIG. 2A). This observation is in line with notion that IgG only interacts with CD32 after cross-linking, and suggests that the abovementioned binding of bovine IgG on immature DC is the result of IgG immune complexes or aggregates.

In an attempt to determine whether the binding of human and bovine IgG on immature DC was CD32 specific, immature DC were pre-treated with a known blocking mouse anti-human CD32 antibody (10 μg/mL; clone FL18.26 from BD Pharmingen). This anti-CD32 pre-treatment clearly reduced the binding of human aggregated IgG (see FIG. 2B). The residual binding of human IgG after blocking with anti-CD32 suggests that other receptors such as FcRN may also play a role in binding of aggregated/complexed IgG (human but also bovine) to human DC.

### Example 2

### Biological Activity of Immunoglobulins on Immature Dendritic Cells

LPS-insensitive (TLR-4low and CD14low) immature DC were exposed to bovine colostrum and purified bovine immunoglobulins with and without trace amounts of LPS from Escherichia coli in an attempt to mimic soluble immune complexes. After 2 days of exposure, DC activation was monitored by examining cytokine production (i.e. IL-10 and TNF-α).

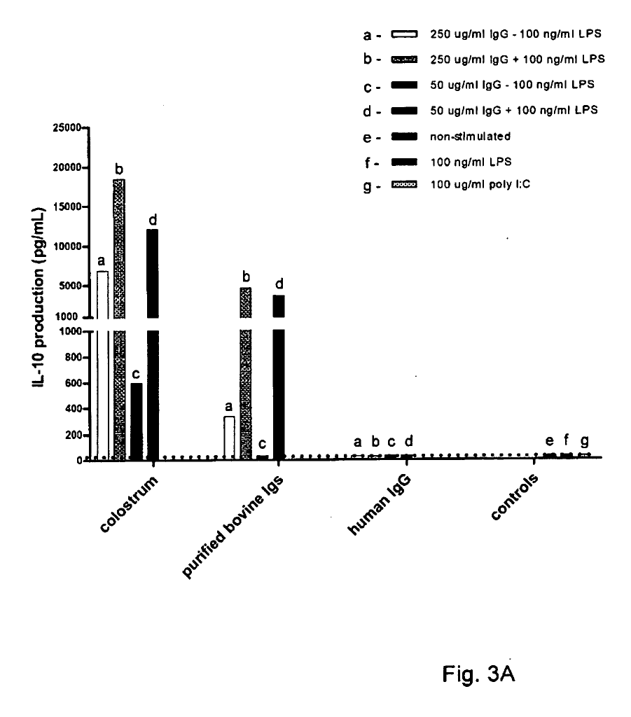

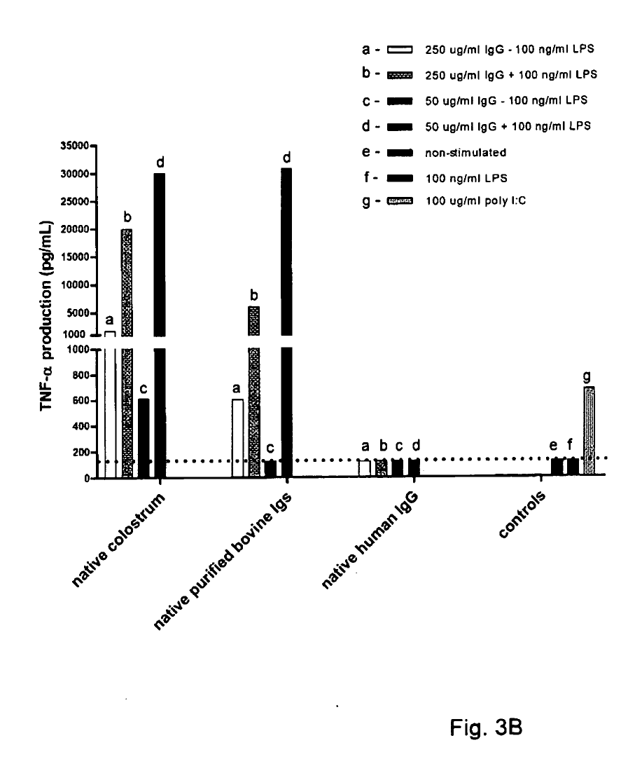

Bovine colostrum strongly activated human DC, which was exemplified by the reproducible high levels of IL-10 and of TNF-α released by immature DC after 2 days treatment (see FIG. 3). This effect was dose dependent (i.e. 250 μg/mL>50 μg/mL). Purified bovine immunoglobulins also demonstrated dose dependent human DC activation (i.e. 250 μg/mL>50 μg/mL).

We have previously demonstrated the presence of bacteria specific bovine antibodies, including against LPS, in milk (see PCT Publication WO2008/127105). The combination of 100 ng/mL LPS with bovine colostrum and especially with purified bovine immunoglobulins resulted in a strong synergistic effect on the IL-10 and TNF-α secretion from human immature DC (see FIG. 3). LPS at 100 ng/ml alone had no affect on cytokine production. This observation indicates that immune complexes consisting of bovine anti-LPS specific immunoglobulins and LPS activate human immature DC via a FcR signalling pathway.

### Example 3

### Survival of Bovine Immunoglobulins in the Human GI Tract

In vitro digestion experiments were performed to determine the effect of digestion in the stomach and small intestine on bovine immunoglobulins. To this aim effects of an infant and adult in vitro digestion protocol on the stability of bovine IgG1 and IgA in colostrum were studied.

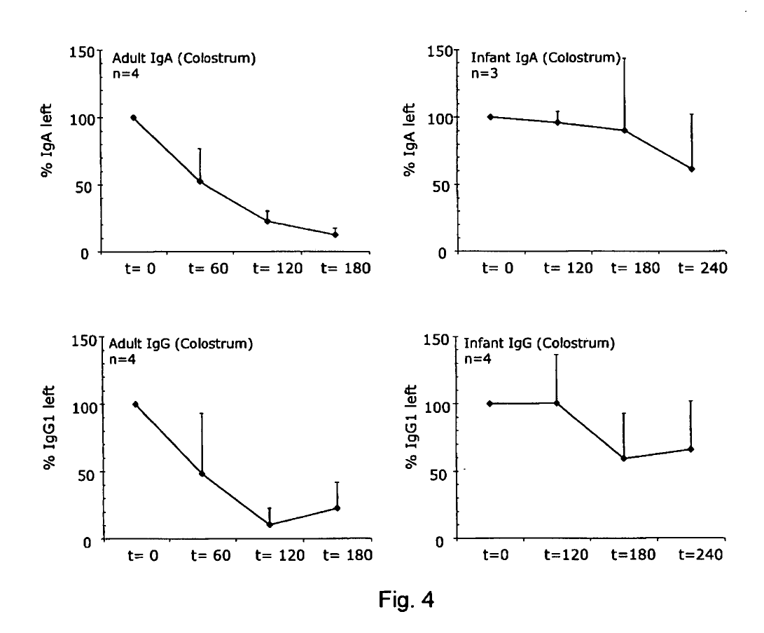

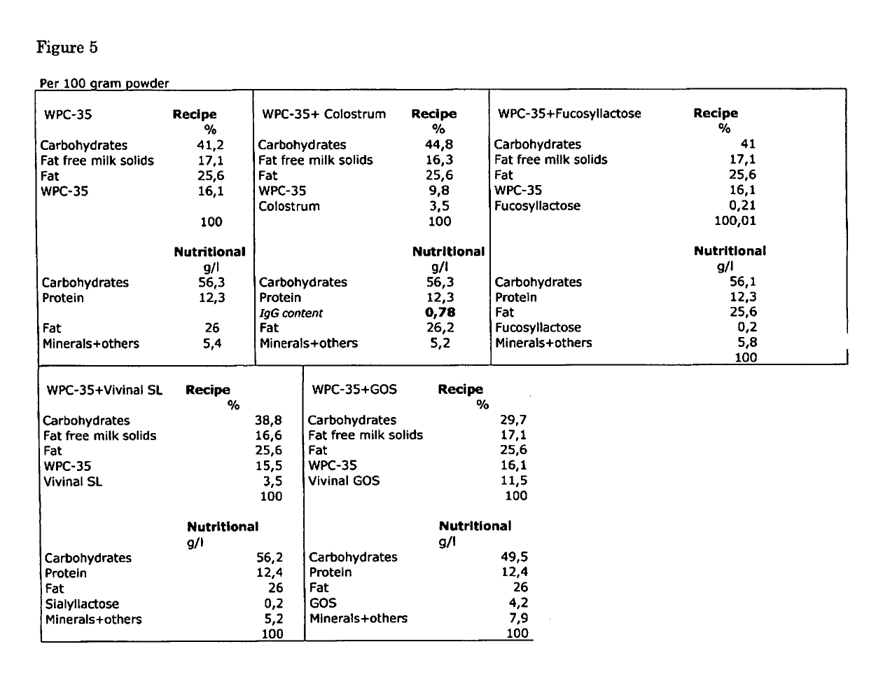

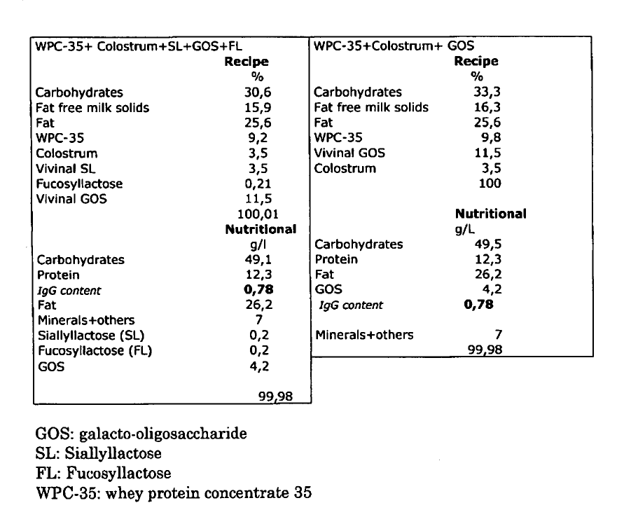

As shown in FIG. 4, the infant digestion protocol left approximately 40-60% of bovine IgG1 and IgA intact after the small intestine treatment. The adult protocol, which is more vigorous, leaves approximately 10-20% IgG1 and IgA intact. In another independent experiment <5% of immunoglobulins survived the adult stomach and small intestine treatment. At least part of the difference seen between the infant and adult conditions may be the result of pH differences. The pH of the gastric phase in young infants is 4 (this is pH 3 in the adult protocol) and the duration of the gastric phase is 1 hour for young infants and to 2 hours for adults. We have recently seen that bovine IgG1 and IgA were stable for 6 hours at pH 4, but were quite sensitive to even short exposure to pH3,5 and lower. Overall, these data indicate that a significant amount of orally ingested immunoglobulins are present throughout the human intestine and can therefore be expected to maintain functionality.

These experiments confirm previously published results. An early study from 1979 demonstrated that active E. coli EPEC-specific Ig or active Ig fragments thereof are present in stools of infants treated with immunoglobulins from immunized cows after passage through the GI tract, and that extracts prepared from these stools can protect mice after against an in vivo challenge with E.coli (Hilpert H., et al., Nestlé Res.News , 134-138. 1974). Likewise, Hilpert et al have previously shown that 10-20% of the immunoglobulins may survive intestinal passage in infants (Hilpert,H., et al. 1987. J. Infect. Dis. 156:158-166), and Pacyna et al have shown that anti-rotaviral immunoglobulin activity can be detected in the stools of children with rotavirus-induced diarrhea (Pacyna,J., et al. 2001. J. Pediatr. Gastroenterol. Nutr. 32:162-167).

Another study showed that approximately 20% of IgG and IgM from colostrum, but not IgA survives through the ilium, in adults (Roos,N., et al. 1995. J. Nutr. 125:1238-1244). This is in line with a study by Kelly et al who described that up to 3% of orally ingested bovine IgG could survive the GI tract passage from mouth to anus in healthy adults (Kelly, C. P., et al. 1997. Antimicrob. Agents Chemother. 41:236-241). When the immunoglobulins were in a capsule that dissolves at pH 6 and higher, even 30% could be detected in the faeces. In individuals that took colostrum after fasting about 2-fold more IgG survived the passage throughout the intestine. In a similar study, a survival of 50% of all orally administered IgG1 was shown to survive troughout the ilium, the most proximal part of the small intestine (Warny,M., et al. 1999. Gut 44:212-217). No enhanced survival was seen by combining the colostrum with antacid to prevent against low pH or omeprazole. Enteric capsules were mostly intact in the ilium, suggesting that immunoglobulins administered in enteric capsules will not be released before entering the colon. These data support our results that a significant amount of orally ingested immunoglobulins are present throughout the infant stomach and small intestine, and can therefore be expected to maintain functionality.

### Example 4

### Measurement of Bovine IgG1, IgM, and IgA in Milk, Whey, Colostrum and Milk-Derived Protein Preparations

ELISA assays were performed as described in Example 1 to measure the amounts of bovine immunoglobulins from various sources including milk. As shown in Table 2, raw milk contains significant levels of IgG.

TABLE 2

| | IgG1 | IgA | IgM | Ratio |
|---|---|---|---|---|
| | (μg/ml) | (μg/ml) | (μg/ml) | IgG1:IgA |
| Protein preparations | | | | |
| beta 535 (20 mg/ml) | 8.2 | 1.4* | <det | 5.7 |
| Viv. Alfa (20 mg/ml) | 7.4 | 2.7* | 3.8# | 2.7 |
| Deminal 90 (20 mg/ml) | 17.4 | 0.9* | 1.3 | 18.8 |
| SPG Albert (20 mg/ml) | 171.8 | 14.6* | <det | 11.8 |
| Hip 45 (20 mg/ml) | 58.6 | 0.4* | <det | 130.7 |
| SPC Leeuwarden (20 mg/ml) | 267.3 | 6.4 | 24.6# | 41.8 |
| Lactive (20 mg/ml) | 807.8 | 44.1* | nd | 18.3 |
| Colostrum powder*** | | | | |
| Colostrum A (20 mg/ml) | 1292.5 | 162.9* | 110.7 | 7.9 |
| Colostrum B (20 mg/ml) | 2387.5 | 153.4* | 140.6 | 15.6 |
| Colostrum C (20 mg/ml) | 1960.0 | 189.1* | 27.9 | 10.4 |
| Whey/milk | | | | |
| Osmose whey Dalfsen | 336.5 | 137.0 | 24.5 | 2.5 |
| Whey A | 76.4** | 25.5* | nd | 3.0 |
| Whey B | 77.7** | 28.2* | nd | 2.8 |
| MaestroA | 80.8 | 22.8 | 7.7# | 3.5 |
| MaestroB | 52.0 | 15.1 | 5.8# | 3.5 |
| Therm milk | 227.0 | 89.8 | 61.6 | 2.5 |
| Raw milk | 213.5 | 118.8 | 57.5 | 1.8 |
| Milk sample 15-5-07 | 212.8 | 151.6* | 84.0 | 1.4 |
| Microfiltr. 50° | 138.0 | 70.4* | 33.4 | 2.0 |
| Microfiltr. 10° | 37.9 | 3.9* | <det | 9.7 |

Measurements of Bovine Immunoglobulins in Whey, Colostrums, Milk and Protein Preparations

Data represent averages from two separate ELISAs except where indicated otherwise.

*—IgA samples from single measurement because standard curve was not OK

**—Single IgG1 measurement

***—Protein content of powders unknown

#—Single measurement, sample in second ELISA below detection

nd —Not determined

Material and Methods Used in the Examples

Purification of Bovine Colostrum-Derived Immunoglobulins

Diluted colostrum sample in 0.75 M (NH₄)₂SO₄ loading buffer (two-fold dilution). Added additional (NH₄)₂SO₄ to obtain a final concentration of 0.75 M (NH₄)₂SO₄. Loaded sample onto an AFFI-T™ column (Kem-en-Tec). Eluted sample with 0.05 M Tris-HCl pH 8.9 elution buffer. Diluted sample with 0.02 M NaPi pH 7.0 loading buffer (four-fold dilution). Loaded sample on a proteinG column (5 ml; Amersham). Eluted sample with 0.1 M glycine-HCl pH 2.7 elution buffer. Neutralized sample with 1 M Tris-HCl pH 9.0 neutralizing buffer. Dialyzed against PBS (3×1L). Filtrated purified Igs sample to obtain sterility (0.2 μm filter). Measured A280 (GeneQuant; Amersham) to determine protein concentration. Stored at 4° C.

Determination of Bovine Subclass IgG1, IgG2, IgA, and IgM in Bovine Colostrum BCPLF200, Purified Bovine Immunoglobulins, and Digestion Samples.

Coated ELISA plate (Corning) with 100 μL/well coating antibodies (i.e. 1.25-2.50 μg/mL sheep anti-bovine IgG, IgA, or IgM (Bethyl) diluted in PBS pH 7.4),Incubated overnight at 4° C. in a humidified environment. Washed (300 μL/well) plate 3× with 0.05% Tween-20 (ICN)/PBS wash buffer. Blocked (200 μL/well) plate with 1:10 block buffer (Roche). Incubated 1 hour at 37° C. in a humidified environment. Washed 3× with wash buffer. Added 100 μL/well standards (bovine reference serum; Bethyl), and bovine colostrum and purified bovine immunoglobulins (diluted in block buffer). Incubated 1 hour at 37° C. in a humidified environment. Washed 3× with wash buffer. Added 100 μL/well detection antibodies conjugated to peroxidase (i.e. 0.02 μg/mL sheep anti-bovine IgG1, IgG2, IgA, or IgM (AbD Serotec) diluted in block buffer).

Incubated 1 hour at 37° C. in a humidified environment. Washed plate 5× with wash buffer. Added 100 μL/well TMB solution (Biosource). Incubated 15 minutes at RT. Stopped reaction by adding (100 μL/well) 1 M H₂SO₄. Measured absorbance at dual wavelength 450 nm/655 nm (655 nm as a reference) using a microplate reader.

Generation and Activation of Human Immature Monocyte-Derived DC

Human monocytes were isolated from huffy coats by density centrifugation on Lymphoprep (1,077 g/ml), followed by density centrifugation over a percoll gradient (three densities: 1,076 g/ml, 1,057 g/ml, and 1,054 g/ml). After washing the cells and resuspending them in serum free medium (CellGro DC medium, CellGenix) supplemented with Gentamycin, the cells were allowed to adhere to the surface of a cell surface flask for 1,5 h, non-adherent cells were removed, and fresh medium was added. 20 ng/mL GM-CSF (PeproTech) and 20 ng/mL IL-4 (PeproTech) was added to adhered monocytes. Monocytes were incubated for 5 days in a 5% CO2 incubator at 37° C. (add ‘fresh’ 20 ng/mL GM-CSF and 20 ng/mL IL-4 on day 2). After 5 days culture immature DC were harvested and adjusted to 1.0×106 DC/mL in CellGro DC medium/gentamycin supplemented with 20 ng/mL GM-CSF and 20 ng/mL IL-4 per polypropylene tube (Greiner). Added 250 μg/mL or 50 μg/mL bovine IgG1 from colostrum and purified bovine immunoglobulins with or without 100 ng/ml LPS from Escherichia coli (serotype 055:B5; Sigma) to DC. In addition, run human IgG (Jackson ImmunoResearch) at 250 μg/mL or at 50 μg/mL with or without 100 ng/ml LPS, and non-stimulated DC and 100 μg/mL polyl:C (Sigma) treated DC (PeproTech) as negative and positive controls respectively. Incubated for 2 days in a 5% CO2 incubator at 37° C. After 2 days, collected supernatant (store at −20° C.) for determining cytokine levels.

Binding of Bovine Colostrum, Purified Bovine Immunoglobulins, and Human IgG on Immature DC

Washed immature DC in PBS/0.1% BSA/0.05% NaN₃ (Acros Organics) at 4° C., and adjusted to 1×105 cells/100 μL PBS/BSA/NaN₃ at 4° C. per tube. Added 250 μg bovine IgG1/mL from colostrum and purified bovine immunoglobulins, and 250 μg native (monomeric) or denatured (20 minutes at 65° C.; to mimic immune complexes) human IgG/mL (Jackson ImmunoResearch). Incubated 30 minutes at 4° C. Washed cells (3×; 3 minutes 500 g) in excess (4 mL/tube) of PBS/BSA/NaN₃ at 4° C. Resuspended to 1×105 cells/100 μL PBS/BSA/NaN₃ at 4° C. Added 10 μl 1:10 sheep anti-bovine IgG (Bethyl), 10 μl 1:10 sheep anti-bovine IgA (Bethyl), and 10 μl 1:10 sheep anti-bovine IgM (Bethyl), or 10 μl 1:10 goat anti-human IgG-FITC (Jackson ImmunoResearch). Incubated 30 minutes at 4° C. Washed cells (3 minutes 500 g) in excess (4 mL/tube) of PBS/BSA/NaN₃ at 4° C. Resuspended to 1×105 cells/100 μL PBS/BSA/NaN₃ at 4° C. Added 10 μl 1:10 donkey anti-sheep IgG-FITC (Jackson ImmunoResearch). Incubated 30 minutes at 4° C. Washed cells (3 minutes 500 g) in excess (4 mL/tube) of PBS/BSA/NaN₃ at 4° C. Resuspended to 1×105 cells/200 μL PBS/BSA/NaN₃/2% formaldehyde at 4° C. Incubated 30 minutes at 4° C. Measured (10,000 events/tube) MFI to determine Ig binding using a flow cytometer (Becton & Dickinson).

Effect of Digestion (Stomach and Small Intestine) on Immunoglobulin Stability

The digestion protocols used for digestion of immunoglobulin containing samples are based on the upper GI-tract conditions in young infants and adults. These two protocols differ in some aspects. The pH of the gastric phase of the digestion of young infants the pH is 4 (this is pH 3 in the adult protocol) and the duration of the gastric phase is 1 hour for young infants and to 2 hours for adults. The pepsin concentration is 80 mg for adults and 28 mg for young infants. The small intestine digestion is the same.

a) Protocol for mimicking digestion in the stomach and small intestine in infants:

1 gram of colostrum dissolved in 50 ml water was incubated batch wise with 20 ml stomach juice (containing 28 mg pepsin; Sigma P-6887) at 37° C. and pH 4.0 for 120 min. After this the pH was increased to pH 6.5 with bicarbonate. Then 25 ml small intestine fluid (containing 100 mg pancreatin: Sigma P-1750) was added and the samples are incubated for another 120 min at 37° C. Directly after the digestion the samples were stored at −20° C. until measurement of IgG1 and IgA.

b) Protocol for mimicking digestion in the stomach and small intestine in adults:

1 gram of colostrum dissolved in 50 ml water was batch wise incubated with 20 ml stomach juice (containing 80 mg pepsin; Sigma P-6887) at 37° C. and pH 3.0 for 60 min. After this the pH was increased to pH 6.5 with bicarbonate. Then 25 ml small intestine fluid (containing 100 mg pancreatin: Sigma P-1750) was added and the samples were incubated for another 120 min at 37° C. After the digestion the samples are stored at −20 ° C. until measurement of IgG1 and IgA.

1-31. (canceled)

32. A method for enhancing and/or inducing an adaptive immune response in an individual, comprising administering to said individual in need thereof an antigen-specific antibody obtained from the milk, milk product, colostrum, or colostrum product of a ruminant.

33. The method of claim 32, wherein said individual is afflicted with or at risk of developing an allergic disease, an autoimmune disease, an infectious disease, and/or an inflammatory disease.

34. The method of claim 32, wherein the antigen of the antigen-specific antibody is present in the individual.

35. The method of claim 32, further comprising conjointly administering to said individual said antigen specific antibody and the antigen of the antigen-specific antibody.

36. The method of claim 32, wherein said administration promotes a non-inflammatory immune response in the respiratory tract or intestine of the individual.

37. The method of claim 32, wherein said administration promotes a non-inflammatory immune response in the respiratory tract or intestine of the individual.

38. The method of claim 32, wherein said ruminant is bovine.

39. The method of claim 32, wherein said antibody is obtained from milk.

40. The method of claim 32, wherein said antibody is administered as a functional food.

41. The method of claim 32, wherein the ruminant has not been immunized with the antigen of the antigen-specific antibody prior to collecting the milk or colostrum.

42. The method of claim 32, wherein said antibody is specific for an allergy antigen.

43. The method of claim 32, wherein the efficient presentation of the antigen to T-cells in said individual is promoted.

44. The method of claim 32, wherein the production of non-inflammatory, antigen-specific IgA in said individual is promoted.

45. The method of claim 32, wherein the dendritic cells are activated to produce one or more cytokines in said individual.

46. A method for delaying the onset or reducing the severity of one or more symptoms of a disorder selected from an allergic disease, an autoimmune disease, an infectious disease, and/or an inflammatory disease in an individual at risk of developing said disease, comprising providing said individual at risk thereof with an antigen-specific antibody obtained from the milk, milk product, colostrum, or colostrum product of a ruminant.

47. The method of claim 46, wherein the individual is conjointly administered said antigen.

48. A method for promoting the efficient presentation of the antigen to T-cells in an individual or for promoting the production of non-inflammatory, antigen-specific IgA in an individual, comprising providing to an individual in need thereof a therapeutic amount of an antigen-specific antibody obtained from the milk, milk product, colostrum, or colostrum product of a ruminant.

49. A method for activating dendritic cells to produce one or more cytokines , preferably IL-10 and/or TNF-α, in an individual comprising providing to an individual in need thereof a therapeutic amount of an antigen-specific antibody obtained from the milk, milk product, colostrum, or colostrum product of a ruminant.

50. Child nutritional composition for enhancing and/or inducing an adaptive immune response comprising antigen-specific antibody obtained from the milk, milk product, colostrum, or a colostrum product of a ruminant

51. Child nutritional composition of claim 50, wherein said antibody is obtained from milk.
